(19) United States
(12) Patent Application Publication
(10) Pub. No.: US 2026/0301862 A1
(43) Pub. Date: Oct. 1, 2026

(54) Systems and Methods for Additive Genomic Sequencing With Cumulative Coverage and Longitudinal Analysis

(71) Applicant: Mohammed Harris, Basking Ridge, NJ (US)

(72) Inventor: Mohammed Harris, Basking Ridge, NJ (US)

(73) Assignee: TimepointDx, Basking Ridge, NJ (US)

(21) Appl. No.: 19/091,693

(22) Filed: Mar. 26, 2025

(51) Int. Cl. G16B 20/20; C12Q 1/6869; G06N 20/00; G16B 30/10; G16B 40/00; G16B 50/30; G16H 10/60; G16H 50/20

(52) U.S. Cl. CPC G16B 20/20; C12Q 1/6869; G06N 20/00; G16B 30/10; G16B 40/00; G16B 50/30; G16H 10/60; G16H 50/20

(57) ABSTRACT

The invention provides systems and methods for additive genomic sequencing through repeated moderate-depth sequencing of biological samples collected at predetermined intervals. The system includes components for sample management, sequencing, data storage, computational processing, and output generation. Samples undergo whole-genome sequencing at approximately 10X coverage or whole-exome sequencing at approximately 30X coverage per time point, typically collected weekly over extended periods. Sequencing datasets from each time point are aligned to a reference genome, merged to generate a cumulative dataset with substantially increased coverage depth, and analyzed to identify genomic variants. The invention further provides computer-implemented methods for integrating temporal genomic data through standardized bioinformatics pipelines with batch effect correction. By tracking allele frequencies across different time points, the system generates longitudinal profiles of genomic changes, enabling detection of rare variants, somatic mutations, and significant temporal trends that would remain undetectable in conventional single-timepoint sequencing approaches.

### FIELD OF THE DISCLOSURE

The present invention relates generally to genomic sequencing technologies and, more particularly, to systems and methods for additive genomic sequencing that accumulate coverage depth across multiple time points, enabling both ultra-deep cumulative coverage and longitudinal tracking of genomic variations for enhanced detection of rare variants and temporal genomic changes.

### BACKGROUND OF THE INVENTION

Next-generation sequencing (NGS) technologies have fundamentally transformed the landscape of genomics over the past two decades, enabling widespread and more cost-effective DNA sequencing in both research and clinical applications (Shendure et al., 2017; Mardis, 2017). Among the most prominent NGS platforms are whole-genome sequencing (WGS) and whole-exome sequencing (WES). WGS targets the entire genome, while WES focuses on the protein-coding regions, which constitute roughly 1-2% of the entire genome yet contain the majority of known disease-associated variants (Brlek et al., 2024; Gilissen et al., 2014). In standard clinical scenarios, a single WGS event might be performed at 30X coverage, while a single WES event might be conducted at 100X coverage. Although these depths often suffice for diagnostic snapshots, they provide limited insight into how genomes evolve over time.

The concept of genomic sequencing has traditionally centered on single or infrequent high-coverage sequencing events. This approach provides a static snapshot of an individual's genetic landscape at a specific moment. In clinical settings, patients typically undergo genomic sequencing once or very few times throughout their lifetime, capturing minimal information about genomic changes that occur over time. This limitation restricts our understanding of dynamic processes such as somatic mutation accumulation, clonal expansion, and genomic responses to environmental factors or therapeutic interventions.

Deeper coverage in genomic sequencing generally enables more sensitive detection of variants, particularly those present at low allele frequencies. However, achieving ultra-deep coverage through a single sequencing event can be prohibitively expensive and logistically challenging. In clinical WGS, 30X coverage is often considered standard, while clinical WES might reach 100X coverage (Brlek et al., 2024; Wetterstrand, 2022). These coverage depths, while adequate for many diagnostic purposes, may miss rare variants or emerging mutations that could have significant clinical implications.

Cancer genomics particularly highlights the limitations of static genomic snapshots.

Tumors are inherently heterogeneous and dynamic, with subclones evolving over time due to selective pressures, including those introduced by therapeutic interventions (Vogelstein et al., 2013; Wan et al., 2017). A single sequencing event captures only the genomic landscape at one moment, potentially missing crucial information about emerging resistant clones or evolving genomic signatures that could inform treatment decisions. Similar challenges exist in tracking infectious diseases, where pathogen genomes can rapidly evolve during infection.

In population health and epidemiological studies, understanding how genetic variants shift in response to environmental exposures, lifestyle changes, or interventions requires repeated genomic assessments. Traditional approaches to sequencing make such longitudinal tracking expensive and computationally challenging (Burton et al., 2014). The inability to efficiently monitor genomic changes over time limits our capacity to establish causal relationships between genetic variations and health outcomes in population studies.

Data integration across multiple sequencing events presents significant computational challenges. Each sequencing run generates massive datasets that must be processed, aligned, and analyzed. When attempting to combine data from multiple time points, issues such as batch effects, variable coverage, and alignment inconsistencies can compromise the integrity of the final analysis (Tarazona et al., 2015; Wandelt et al., 2012). Standard bioinformatics pipelines are typically designed for processing single sequencing events rather than systematically integrating data across time points.

Technical advancements have improved sequencing efficiency and reduced costs (Check Hayden, 2014), but the conceptual framework of genomic sequencing has remained largely focused on static assessments. Hardware-accelerated bioinformatics pipelines like DRAGEN (Illumina, 2023) and sophisticated variant callers have enhanced our ability to process and analyze genomic data, yet the potential for longitudinal genomic monitoring remains largely untapped.

Current approaches to variant detection and tracking over time often involve completely separate sequencing events with independent analyses, missing opportunities to leverage cumulative coverage for enhanced sensitivity. The absence of standardized methods for integrating sequencing data across time points creates inefficiencies and inconsistencies in longitudinal genomic studies.

While some research has explored combining multiple sequencing datasets to improve variant detection (Choi et al., 2009; Cummings et al., 2020), a comprehensive methodology for systematically accumulating coverage while maintaining temporal resolution has not been fully developed. The potential benefits of such an approach include not only deeper effective coverage but also the ability to track genomic changes over time-a combination that could revolutionize how we understand and respond to genomic variations in both research and clinical contexts.

The limitations of current sequencing paradigms highlight the need for innovative approaches that can provide both deeper coverage and longitudinal insights into genomic variations. These insights could significantly advance our understanding of disease progression, treatment response, and population health dynamics.

### SUMMARY OF THE INVENTION

In some aspects, the present invention provides systems and methods for additive genomic sequencing that fundamentally transform how genetic information is gathered and analyzed over time. Rather than relying on single high-coverage sequencing events, this invention introduces a paradigm of repeated moderate-depth sequencing at predetermined intervals, followed by computational integration of the resulting data to achieve both ultra-deep cumulative coverage and longitudinal insights into genomic changes.

In certain embodiments, the invention comprises a comprehensive system for additive genomic analysis that includes integrated components for sample management, sequencing, data storage, computational processing, and output generation. The system is designed to track multiple biological samples collected from a subject at predetermined time intervals and process them through a standardized workflow to generate a cumulative genomic profile with unprecedented depth and temporal resolution.

Some aspects of the invention relate to the sequencing component, which is configured to perform moderate-depth sequencing on each biological sample. This may include whole-genome sequencing at approximately 10X coverage per time point (can be 1-10X) or whole-exome sequencing at approximately 30X coverage per time point (can be 1-100X), depending on the specific application and research objectives.

Further aspects involve the predetermined time intervals for sample collection, which may be customized according to the particular research or clinical scenario. In exemplary implementations, samples may be collected weekly, resulting in 52 sequencing events per year and yielding cumulative coverage of 520X for WGS or 1560X for WES—far exceeding the depth achievable through conventional single-time-point approaches.

In additional aspects, the computational processing component aligns the sequencing data from each time point to a reference genome and merges the aligned data to generate a cumulative dataset with dramatically increased coverage depth. This component further identifies genomic variants using the cumulative dataset and tracks temporal changes in variant allele frequencies across time points, providing a dynamic view of genomic evolution.

Some embodiments incorporate hardware-accelerated pipelines utilizing technologies such as DRAGEN to efficiently process the substantial volumes of data generated through repeated sequencing events. These accelerated pipelines enable faster alignment, variant calling, and integration of multi-timepoint data, making the additive approach more practical and accessible.

In particular aspects, the invention implements sophisticated batch effect correction techniques to minimize variability between sequencing runs conducted at different time points. These corrections are essential for ensuring that the cumulative dataset accurately reflects biological reality rather than technical artifacts introduced during the sequencing process.

Other aspects relate to the method for additive genomic sequencing, which comprises obtaining a plurality of biological samples from a subject at predetermined time intervals, performing sequencing on each sample, aligning the resulting datasets to a reference genome, merging them to generate a cumulative sequencing dataset, and analyzing this dataset to identify genomic variants present in the subject.

Further embodiments describe specialized analytical approaches for detecting rare variants with allele frequencies below the detection thresholds of individual sequencing datasets. By leveraging the enhanced sensitivity provided by cumulative coverage, the invention enables identification of low-frequency variants that would remain invisible to conventional sequencing methods.

In some implementations, the invention includes tracking the emergence and evolution of somatic mutations across the predetermined time intervals, providing crucial insights for applications such as cancer monitoring, where the genomic landscape can change rapidly in response to treatment or other selective pressures.

Even further aspects relate to the computer-implemented method for integrating temporal genomic data, which processes each sequencing dataset through a standardized bioinformatics pipeline to generate aligned read data and variant call data for each time point before aggregating them into a comprehensive cumulative profile.

In more detailed aspects, the standardized bioinformatics pipeline comprises multiple sequential steps including demultiplexing, quality control, alignment, duplicate removal, and base quality recalibration—all performed consistently across time points to ensure compatibility during data integration.

Some embodiments describe the variant calling process, which may utilize a combination of algorithms such as DRAGEN, GATK HaplotypeCaller and FreeBayes to maximize sensitivity and specificity in identifying genomic variants within the cumulative dataset.

Further aspects involve applying statistical methods to identify significant temporal trends in variant allele frequencies, enabling researchers and clinicians to distinguish between random fluctuations and meaningful patterns that may have implications for disease progression or treatment response.

In certain implementations, the invention includes integrating the longitudinal profile of genomic changes with clinical data to identify correlations between genetic variations and disease progression, creating a more comprehensive framework for precision medicine applications.

Other aspects describe secure data management approaches, including storing intermediate data files in distributed cloud-based storage systems with version control, ensuring data integrity and accessibility throughout the additive sequencing process.

Further embodiments relate to the output component, which generates comprehensive reports and visualizations that display both cumulative coverage analysis and the evolution of variant allele frequencies across different time points, making complex genomic data more interpretable for researchers and clinicians.

In some aspects, the invention enables detection of emerging resistance mutations or evolving clonal populations in cancer patients, providing early warning of treatment failure and allowing for timely intervention before clinical symptoms manifest.

Even further aspects relate to applications in population health, where additive sequencing can reveal subtle genomic shifts in response to environmental exposures, lifestyle modifications, or public health interventions, offering valuable insights for epidemiological research and public health policy.

In the broadest aspects, the invention represents a fundamental shift in genomic analysis from static snapshots to dynamic monitoring, opening new avenues for understanding the temporal dimension of genomic changes and their implications for human health and disease.

### BRIEF DESCRIPTION OF THE DRAWINGS

The accompanying drawings, which are incorporated in and constitute a part of this specification, illustrate embodiments of the invention and, together with the description, serve to explain the principles of the invention. In the drawings:

FIG. 1 is a high-level workflow diagram illustrating the additive sequencing process from sample collection through data integration.

FIG. 2 is a computational pipeline diagram showing the processing steps for genomic data at each time point and their integration into a cumulative dataset.

FIG. 3 is a system architecture diagram depicting the relationships between the components of the additive genomic sequencing system.

FIG. 4 is a longitudinal analysis diagram illustrating how variant detection sensitivity improves with cumulative coverage and how genomic changes can be tracked over time.

### DETAILED DESCRIPTION OF PREFERRED EMBODIMENTS

The following description of preferred embodiments refers to the accompanying drawings, which illustrate specific embodiments of the invention. Other embodiments having different structures and operations do not depart from the scope of the present invention. The same reference numbers may be used in the drawings and the following description to refer to the same or like parts.

As used herein, the terms “comprising,” “including,” “containing,” “characterized by,” and grammatical equivalents thereof are inclusive or open-ended and do not exclude additional, unrecited elements or method steps, unless otherwise stated. Other than in the operating examples, or where otherwise indicated, all numbers expressing quantities, processing parameters, assessment scores, and so forth used in the specification and claims are to be understood as being modified in all instances by the term “about,” meaning within a reasonable range of the indicated value. The terms “a” and “an” refer to one or more of the elements described, whereas the term “plurality” refers to two or more of the elements described, unless the context clearly indicates otherwise.

The Additive Genomic Sequencing system and method (also known as Aggregated Longitudinal Sequencing) described herein provide novel solutions for generating ultra-deep coverage genomic data through cumulative sequencing across multiple time points. The invention incorporates advanced sample collection techniques, standardized sequencing protocols, and sophisticated computational integration methodologies to enable highly sensitive variant detection and longitudinal monitoring of genomic changes. The following detailed description, along with the accompanying drawings, provides a comprehensive understanding of the various embodiments and aspects of the invention.

This invention addresses the challenges of obtaining deep coverage genomic data and capturing temporal genomic changes in a systematic and cost-effective manner. By leveraging repeated moderate-depth sequencing at predetermined intervals, the invention eliminates the limitations of single-timepoint sequencing approaches, thereby improving variant detection sensitivity and enabling monitoring of genomic evolution. The system and method further integrate advanced technologies, including hardware-accelerated bioinformatics pipelines and sophisticated batch effect correction techniques, to enhance the accuracy and utility of the generated genomic profiles for research and clinical applications.

The present invention provides a comprehensive system and method for additive genomic sequencing, comprising: a sample management component configured to track multiple biological samples collected at predetermined time intervals; a sequencing component for performing moderate-depth sequencing on each sample; a computational processing component for aligning, merging, and analyzing sequencing data across time points; and an output component for generating reports that include both cumulative coverage analysis and longitudinal tracking of genomic variations.

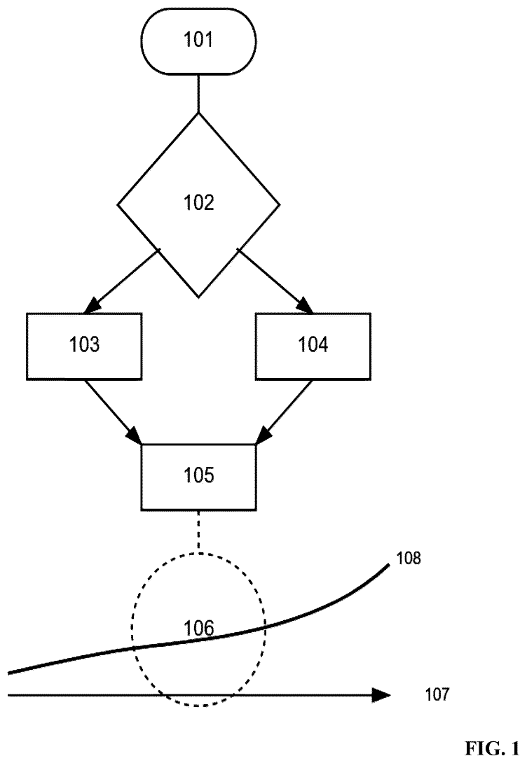

Firstly, FIG. 1 illustrates a high-level workflow diagram of an exemplary additive sequencing process according to one embodiment of the present invention. The workflow represents a systematic approach to generating high-depth genomic data through cumulative sequencing across multiple time points. Element 101 depicts the initial sample collection process, which may involve obtaining biological material such as whole blood, buccal swabs, or tissue samples from a subject. These samples may be collected at predetermined intervals, such as weekly, monthly, or quarterly, depending on the specific research or clinical requirements. In some embodiments, the sample collection process may incorporate standardized protocols to minimize variability across time points.

Secondly, the collected samples proceed to element 102, which represents the library preparation phase. This critical step may involve DNA extraction, fragmentation, adapter ligation, and amplification procedures that prepare the genetic material for sequencing. In certain implementations, library preparation protocols may be specifically optimized for consistency across multiple time points, as batch effects at this stage could potentially impact downstream analysis. The diamond shape of element 102 signifies its role as a decision point in the workflow, where the prepared libraries may be directed toward different sequencing approaches based on research objectives or available resources.

Following library preparation, the workflow branches into two potential sequencing paths, represented by elements 103 and 104. Element 103 depicts a low-pass whole-genome sequencing (WGS) approach, which may typically operate at approximately 10X coverage per time point. This approach allows for broad genomic surveillance at moderate depth, capturing information across the entire genome while managing costs for repeated sequencing events. In a non-limiting embodiment, low-pass WGS may be particularly suitable for tracking structural variants, copy number alterations, or genome-wide patterns that do not require the high resolution of targeted approaches.

In parallel, element 104 represents a high-pass whole-exome sequencing (WES) approach, which may typically provide approximately 30X coverage per time point for the protein-coding regions of the genome. This focused approach delivers deeper coverage of exonic regions, where many disease-relevant variants are located, while still enabling the longitudinal monitoring capabilities central to the additive methodology. In some implementations, researchers might employ either WGS or WES exclusively, while other applications may benefit from implementing both approaches in complementary fashion.

The data from both sequencing paths converges at element 105, which represents the data acquisition phase. This step may encompass the initial processing of raw sequencing output, including demultiplexing of pooled samples and preliminary quality assessment. In certain embodiments, this phase may include conversion of raw sequencing output (such as BCL files) into FASTQ format, along with quality filtering to remove low-quality reads or adapter sequences that could compromise downstream analysis. The rectangular shape of element 105 signifies its role as a processing step that standardizes the raw data from either sequencing approach into a format suitable for subsequent integration.

The workflow proceeds to element 106, depicted as a circle representing the data integration phase. This central aspect of the invention may involve merging aligned reads and variant calls from the current time point with those from previous time points to generate a cumulative dataset of progressively increasing coverage depth. The dashed outline indicates the iterative nature of this process, as each new sequencing event contributes additional data to the cumulative pool. In exemplary implementations, specialized computational methods may be employed to handle batch effects, ensure consistent alignment across time points, and properly weight evidence for variant calls when combining data from multiple sequencing events.

Element 107 represents the temporal dimension of the additive sequencing approach, illustrating how multiple sequencing events are distributed across time. This element emphasizes the longitudinal nature of the invention, which fundamentally distinguishes it from conventional single-timepoint sequencing methodologies. In some embodiments, the timing between sequential sequencing events may be adjusted based on the rate of expected genomic change, with rapidly evolving systems (such as cancer or acute infections) potentially requiring more frequent sampling than stable conditions.

Further, element 108 depicts the cumulative coverage curve that results from the additive approach, showing how sequencing depth increases progressively as data from multiple time points is integrated. This curve illustrates a key advantage of the invention: the ability to achieve ultra-deep effective coverage through the aggregation of multiple moderate-depth sequencing events. In exemplary implementations, this cumulative depth may enable the detection of low-frequency variants that would remain invisible to conventional sequencing approaches, while simultaneously providing temporal information about when these variants first appeared and how their frequencies changed over time. The shape of the curve may vary depending on factors such as sequencing depth per time point, total number of time points, and the consistency of coverage across the genome or exome.

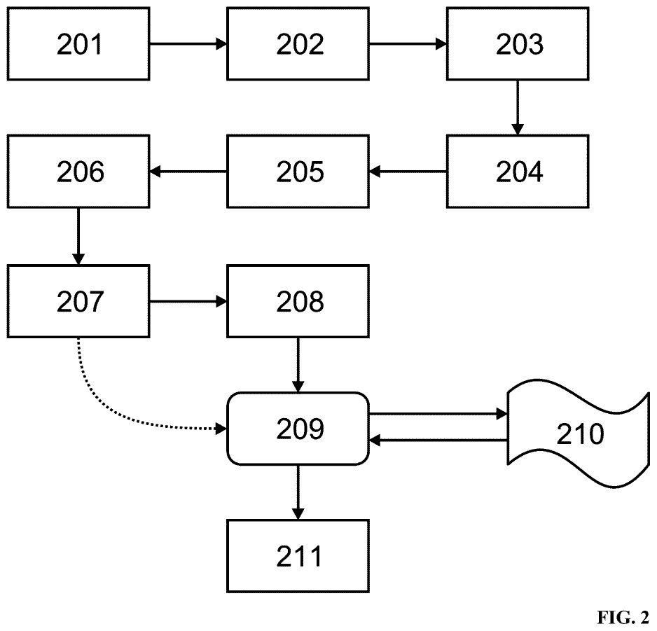

On the other hand, FIG. 2 provides a computational pipeline diagram illustrating an exemplary implementation of the data processing workflow for additive genomic sequencing according to one embodiment of the present invention. Element 201 represents the input data, which may comprise raw sequencing files generated from biological samples at each time point. These input files may typically be in the form of BCL (base call) files or similar formats produced directly by the sequencing instruments. In some embodiments, these input files may be accompanied by metadata that tracks important details such as sample identifiers, collection dates, and sequencing run parameters to ensure proper integration with previously processed data.

In certain implementations, the raw input data proceeds to element 202, which represents the demultiplexing process. This step may involve the separation of pooled sequencing data into individual sample files based on unique barcode sequences that were incorporated during library preparation. Demultiplexing may be performed using standard bioinformatics tools such as Illumina's bc12fastq or similar software packages. The effectiveness of this separation process may influence downstream analysis quality, as improper demultiplexing could potentially lead to sample cross-contamination and subsequent errors in variant calling.

Following demultiplexing, the workflow advances to element 203, representing the quality control phase. During this stage, sequencing reads may be evaluated for various quality metrics such as base quality scores, GC content, sequence duplication rates, and adapter content. In an exemplary embodiment, tools such as FastQC or MultiQC might be employed to generate comprehensive quality reports that help identify potential issues with the sequencing data. Quality filtering may be applied to remove or trim low-quality bases and adapter sequences that could otherwise compromise alignment accuracy and variant detection sensitivity in subsequent steps.

The quality-controlled reads then proceed to element 204, the alignment phase, where the processed sequences may be mapped to a reference genome. This critical step may utilize established alignment algorithms such as BWA-MEM, HISAT2, or hardware-accelerated solutions like DRAGEN. In some implementations, alignment parameters might be specifically optimized and standardized across all time points to ensure consistency in how reads are positioned against the reference sequence, which may be particularly important when merging data from multiple sequencing events.

In a non-limiting embodiment, the aligned reads move to element 205, the duplicate removal process. This step may involve identifying and marking or removing duplicate read pairs that likely originated from the same DNA fragment during PCR amplification in the library preparation phase. Duplicate removal tools such as Picard MarkDuplicates might be employed to address this issue, potentially improving the accuracy of subsequent variant calling by preventing artificial inflation of coverage at certain genomic positions.

The workflow may continue to element 206, which represents base quality score recalibration. This phase may involve adjusting the quality scores assigned to individual bases in each read based on empirical error patterns observed in the data, potentially correcting for systematic biases introduced by the sequencing platform. In certain implementations, this recalibration might be performed using tools from frameworks such as the Genome Analysis Toolkit (GATK), applying machine learning approaches to model error probabilities more accurately.

Element 207 depicts the variant calling phase, where genomic variations may be identified by comparing the processed reads to the reference genome. This step might employ various algorithms such as GATK HaplotypeCaller, FreeBayes, DRAGEN, or other specialized callers optimized for different types of variants. In some embodiments, variant calling might be performed independently at each time point before integration, while in other implementations, aligned reads might be merged first before a single variant calling step is executed on the combined dataset.

The exemplary pipeline then proceeds to element 208, representing coverage calculation. This component may analyze the depth and breadth of sequencing coverage across the genome or targeted regions, generating metrics that help evaluate the completeness and reliability of the sequencing data. Coverage statistics may be particularly important in the additive sequencing context, as they help track how the cumulative depth increases with each additional time point and identify regions that might benefit from targeted follow-up in subsequent sequencing events.

A particularly significant aspect of the computational pipeline is represented by element 209, the data merging component. This step may involve sophisticated algorithms that integrate sequencing data from the current time point with previously processed data from earlier time points. In certain implementations, this integration might occur at the level of aligned reads (BAM files), while in others, it might involve combining variant calls (VCF files) with appropriate statistical frameworks to resolve potentially conflicting evidence across time points.

Element 210 represents the previous data store, which may serve as a repository for sequencing results from all preceding time points. This component might be implemented as a secure database or file system that maintains the integrity and provenance of accumulated genomic data, potentially including both raw and processed files to enable reanalysis if improved methods become available. The bidirectional arrow connecting elements 209 and 210 indicates that the merging process both draws from and contributes to this longitudinal data repository.

Further still, element 211 depicts the final analysis phase, where the integrated data may undergo comprehensive interpretation to extract biologically and clinically relevant insights. This step might involve annotation of variants, prioritization of potentially significant findings, and specialized analyses such as clonality assessment or temporal trend identification that leverage the unique longitudinal dimension of additive sequencing data. Element 212, the time points indicator, emphasizes the iterative nature of the entire process, showing how the pipeline may be repeatedly executed for each new sequencing event, with each iteration contributing to an increasingly comprehensive genomic profile.

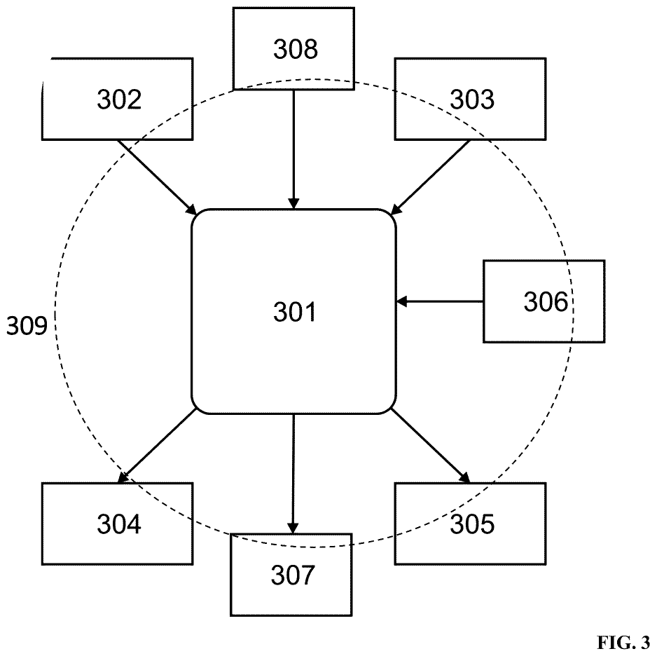

The embodiment of FIG. 3 illustrates a system architecture diagram that depicts the structural organization and interrelationships among the various components of an exemplary additive genomic sequencing system according to one embodiment of the present invention. At the center of the diagram, element 301 represents the central system core, which may serve as the primary integration hub that coordinates the activities of all other components. This core element may comprise a server-based application or distributed computing framework that maintains the overall system state, orchestrates workflows, and manages communication between specialized subsystems. In some implementations, the central system core may include middleware components that standardize data formats and protocols across the various elements of the architecture.

Secondly, element 302 depicts the sample management component, which may be responsible for tracking biological specimens from collection through processing. This component may include features for sample registration, barcode generation, chain-of-custody documentation, and integration with laboratory information management systems (LIMS). In certain embodiments, this component might incorporate scheduling functionality to coordinate regular sample collection at the predetermined intervals required for longitudinal monitoring, potentially generating automated reminders for clinical staff or research subjects when new samples are due to be collected.

The sequencing component, represented by element 303, may encompass the instrumentation and support systems required to perform genomic sequencing at each time point. This may include sequencing platforms configured for either low-pass whole-genome sequencing or high-pass whole-exome sequencing, depending on the specific research or clinical objectives. In some implementations, this component might include automated library preparation systems to enhance consistency across multiple sequencing events, as well as quality monitoring systems that provide real-time feedback on sequencing runs to detect and address potential issues promptly.

In a non-limiting embodiment, element 304 represents the data storage component, which may provide secure, scalable infrastructure for housing the substantial volumes of data generated through repeated sequencing events. This component might implement tiered storage architectures that balance accessibility and cost-effectiveness, potentially utilizing high-performance storage for active analysis and lower-cost archival solutions for long-term retention. The storage component may incorporate robust backup mechanisms and disaster recovery capabilities to protect the integrity of accumulated genomic data, which becomes increasingly valuable as the temporal dimension expands.

Element 305 depicts the computational processing component, which may comprise high-performance computing resources dedicated to executing the bioinformatics pipelines required for data analysis at each time point and integration across the longitudinal series. In certain implementations, this component might leverage cloud-based or hybrid infrastructure to provide elastic scalability as data volumes grow over time. The bidirectional connections between elements 304 and 305 illustrate the continuous exchange of data between storage and processing components throughout the analytical workflow.

The embodiment shown in FIG. 3 may include a hardware acceleration component, represented by element 306, which could provide specialized computational resources optimized for genomic analysis. This might include field-programmable gate arrays (FPGAs) or application-specific integrated circuits (ASICs) that accelerate particular steps in the analysis pipeline, such as read alignment or variant calling. In some implementations, this component might incorporate DRAGEN technology or similar hardware-accelerated genomic analysis platforms to significantly reduce processing time while maintaining high accuracy, which may be particularly valuable when dealing with the large cumulative datasets generated through additive sequencing.

Element 307 represents the output component, which may be responsible for generating reports, visualizations, and other deliverables that communicate the results of additive sequencing analysis to researchers, clinicians, or other stakeholders. This component might include interactive dashboards for exploring longitudinal trends in genomic variants, comparative views that highlight changes between time points, or specialized visualizations that illustrate how cumulative coverage enhances variant detection sensitivity. In certain embodiments, this component might also provide interfaces for exporting data to external systems for specialized analyses or integration with clinical decision support frameworks.

Even further, elements 308 and 309 represent external systems and the security layer, respectively. Element 308 may indicate various third-party platforms or databases that exchange information with the additive sequencing system, such as electronic health records, variant interpretation resources, or population genomics repositories. Element 309, depicted as an enclosing ellipse, represents the comprehensive security infrastructure that protects sensitive genomic data throughout the system. This may include encryption for data at rest and in transit, fine-grained access controls, audit logging mechanisms, and compliance features aligned with relevant regulations such as HIPAA or GDPR, providing essential safeguards for the privacy and confidentiality of longitudinal genomic information.

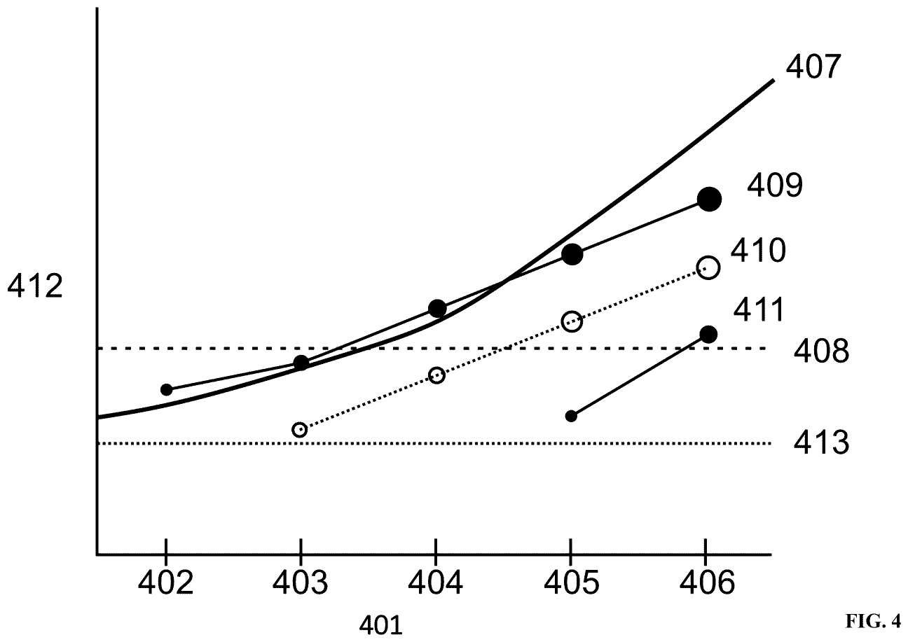

Further still, FIG. 4 presents a longitudinal analysis diagram that illustrates the temporal aspects of variant detection and tracking in the additive sequencing methodology. This visualization provides a graphical representation of how cumulative coverage evolves over time and its impact on the detection of genomic variants. Element 401 depicts the horizontal timeline that forms the foundation of the diagram, representing the progression of sequential time points at which samples are collected and sequenced. This timeline may correspond to days, weeks, months, or other intervals depending on the specific implementation of the additive sequencing approach, with the spacing potentially adjusted to reflect the actual temporal distance between sampling events.

Along the timeline, elements 402, 403, 404, 405, and 406 represent distinct time points at which individual sequencing events may occur. In exemplary implementations, these might correspond to weekly blood draws from a cancer patient undergoing treatment, monthly sampling in a population health study, or other recurring collection schedules tailored to the research or clinical context. Each of these time points may generate its own dataset with moderate coverage depth, which then contributes to the cumulative profile. In some applications, the sampling frequency might be adjusted dynamically based on observed genomic changes, with intervals potentially shortened during periods of rapid evolution or extended during periods of stability.

Element 407 illustrates the cumulative coverage curve, which demonstrates how sequencing depth increases progressively as data from multiple time points is integrated. This curve may typically show a steady upward trajectory, reflecting the additive nature of the approach, though the exact shape might vary depending on factors such as the consistency of coverage across sequencing events. In certain implementations, the curve might exhibit a more pronounced upward slope in earlier time points, potentially reflecting initial rapid gains in coverage that may gradually stabilize as deeper regions approach saturation. The cumulative nature of coverage represents a fundamental advantage of the additive approach, potentially enabling detection of variants present at frequencies below the sensitivity threshold of any individual sequencing event.

In the exemplary diagram, element 408 represents the variant detection threshold, which may indicate the minimum allele frequency at which variants can be reliably identified given the current cumulative coverage depth. This threshold may typically decrease over time as coverage accumulates, reflecting the enhanced sensitivity that results from integrating data across multiple sequencing events. In some implementations, this threshold might be calculated based on statistical models that account for sequencing error rates, coverage distribution, and other technical parameters that influence variant calling confidence, potentially adapting to the specific characteristics of each genomic region.

Elements 409, 410, and 411 illustrate the tracking of individual variants across multiple time points, demonstrating how allele frequencies may change over time and how detection sensitivity improves with cumulative coverage. Element 409 may represent a variant that is present at all time points but becomes more confidently characterized as coverage accumulates. In certain applications, such as cancer monitoring, this might correspond to a founder mutation present in the majority of tumor cells, which remains detectable throughout the monitoring period. The increasing size of the circles along this trajectory may represent growing confidence in the variant characterization as additional evidence accumulates across time points.

Element 410 depicts a variant with a different pattern, potentially representing a subclonal mutation that fluctuates in frequency over time. In oncological applications, such a pattern might indicate a mutation associated with a treatment-responsive subclone that initially decreases in frequency but later reemerges, potentially signaling developing resistance. The dashed line connecting these observations may indicate a level of uncertainty in the exact trajectory between sampling points, acknowledging that genomic changes might occur during intervals between sequencing events. This pattern illustrates how the longitudinal dimension of additive sequencing may reveal dynamic genomic processes that would remain invisible in static, single-timepoint approaches.

Element 411 represents a variant that emerges only in later time points, potentially corresponding to a newly acquired mutation that was not present or was below detection limits in earlier samples. In clinical applications, such emerging variants might represent particularly important findings, potentially indicating disease progression, treatment resistance, or other significant biological changes that warrant intervention. The ability to precisely identify when new variants first appear may provide valuable insights into the timing and potential triggers of genomic evolution, information that cannot be obtained from conventional single-timepoint sequencing approaches.

Element 412 represents the vertical axis, which may correspond to allele frequency or other quantitative measures of variant abundance. This axis provides context for interpreting the patterns of variant detection and tracking illustrated in the diagram. In some implementations, this axis might use a logarithmic scale to better visualize both high-frequency and low-frequency variants simultaneously, reflecting the wide dynamic range of allele frequencies that may be encountered in applications such as cancer genomics or population studies.

Element 413 depicts the single sample detection limit, which may represent the lowest allele frequency at which variants can be reliably detected in any individual sequencing event considered in isolation. This horizontal line illustrates a key limitation of conventional single-timepoint sequencing approaches, as variants present below this threshold would typically be missed without the enhanced sensitivity provided by cumulative coverage. The position of elements 410 and 411 relative to this line demonstrates how the additive approach may enable detection of variants that would remain invisible to standard sequencing methods, potentially including clinically significant low-frequency mutations.

The overall arrangement of elements in FIG. 4 may illustrate multiple complementary benefits of the additive sequencing approach. Beyond simply accumulating coverage to enhance detection sensitivity, the temporal dimension enables tracking of how variant frequencies evolve over time, potentially revealing important patterns such as clonal expansion, treatment response, or resistance development. The integration of these capabilities may be particularly valuable in complex applications such as cancer monitoring, where both the presence of specific mutations and their quantitative trajectories may provide important clinical insights. Additionally, the continuing accumulation of coverage may progressively reveal the broader landscape of low-frequency variants, potentially including rare subclones or mosaic mutations that might have implications for treatment planning or prognosis.

The ability to distinguish between pre-existing low-frequency variants that become detectable as coverage accumulates and genuinely new mutations that arise during the monitoring period represents a unique capability of the additive sequencing approach. This distinction, which cannot be made with conventional single-timepoint deep sequencing, may be critical for accurate interpretation of genomic findings in both research and clinical contexts. By providing both enhanced sensitivity through cumulative coverage and temporal resolution through sequential sampling, the methodology illustrated in FIG. 4 may offer a more comprehensive and nuanced view of genomic dynamics than conventional approaches.

In a non-limiting embodiment, the additive genomic sequencing system may incorporate mobile sample collection devices that enable remote acquisition of biological specimens without requiring subjects to visit clinical facilities. These devices may include specialized blood collection systems that stabilize nucleic acids for extended periods, potentially allowing samples to be shipped at ambient temperature to centralized sequencing facilities. Other examples of remote and point of care blood collection devices may include upper-arm push-button devices such as Tasso+, Tasso Mini, Yourbio Health TAP Micro/Select, RedDrop Dx One, and LetsGetChecked Impress. This approach may broaden accessibility to longitudinal genomic monitoring for subjects in rural or underserved areas.

The embodiment of FIG. 1 may be extended to include branched sequencing pathways wherein certain samples undergo additional specialized analyses based on findings from the primary sequencing approach. For example, if certain variants of interest are detected in the standard low-pass WGS data, the system may automatically trigger targeted deep sequencing of specific genomic regions to obtain higher resolution information while maintaining the efficiency of the overall additive framework.

In another embodiment, the computational pipeline depicted in FIG. 2 may incorporate artificial intelligence components that adaptively optimize analytical parameters based on accumulating data. These machine learning models may continuously refine variant calling thresholds, alignment strategies, and batch correction methods as more time points are integrated, potentially improving sensitivity and specificity beyond what could be achieved with static analytical approaches.

Some embodiments may implement hybrid sequencing approaches that combine short-read and long-read technologies within the additive framework. For instance, routine monitoring might employ cost-effective short-read sequencing at frequent intervals, while less frequent long-read sequencing events might be interspersed to resolve complex structural variants or repetitive regions that are challenging to characterize with short reads alone. The integrated computational pipeline may leverage the complementary strengths of both technologies.

Exemplary implementations may include specialized algorithms for detecting episodic genomic events that occur between sampling intervals. These approaches may analyze patterns of subclonal evolution to infer the likely timing of mutation emergence or selection events, potentially providing insights into genomic dynamics even during periods when direct measurements are not available. Such computational extrapolation may be particularly valuable when sampling frequency is limited by practical constraints.

The embodiment of FIG. 3 may be expanded to include a federated architecture that enables secure data sharing across multiple research or clinical sites while maintaining privacy protections. This distributed approach may facilitate larger-scale longitudinal studies by allowing separate institutions to contribute time-series genomic data without centralizing sensitive information, potentially accelerating discovery while addressing regulatory and ethical concerns.

In certain implementations, the system may incorporate automated scheduling algorithms that dynamically adjust sampling intervals based on observed rates of genomic change. For subjects exhibiting rapid genomic evolution, such as cancer patients experiencing disease progression, the system might recommend more frequent sampling to capture important changes. Conversely, for subjects with stable genomic profiles, sampling intervals might be extended to optimize resource utilization.

An alternative embodiment may integrate multiomic data streams alongside genomic sequencing, tracking changes in transcriptomes, proteomes, metabolomes, or epigenomes in parallel with genomic variants. This comprehensive approach may provide contextual information about the functional implications of detected variants, potentially revealing how genomic changes translate into altered cellular processes over time and across multiple molecular levels.

The embodiment of FIG. 4 may be enhanced with predictive modeling capabilities that forecast likely genomic trajectories based on early time points. These predictive models may leverage patterns observed in similar cases to anticipate the emergence of resistance mutations or disease progression markers, potentially enabling preemptive intervention before clinically significant changes occur. Such forecasting approaches might be particularly valuable in therapeutic monitoring applications.

In some implementations, the additive sequencing system may incorporate sample fractionation techniques that enable separate analysis of distinct cell populations within a single biological specimen. For example, in oncology applications, circulating tumor cells might be isolated from peripheral blood and sequenced separately from leukocytes, potentially providing more specific information about tumor evolution while leveraging the convenience and minimal invasiveness of liquid biopsies.

The embodiment of the computational pipeline may include specialized algorithms for distinguishing true somatic changes from age-related clonal hematopoiesis when monitoring cancer patients through blood-based samples. These approaches may leverage the temporal dimension of additive sequencing to identify different evolutionary patterns characteristic of malignant versus benign clonal expansions, potentially improving the specificity of liquid biopsy monitoring.

In certain implementations, the system may incorporate reference materials with known variant profiles that are processed alongside patient samples at regular intervals. These controls may enable ongoing calibration of the entire workflow, from sample collection through sequencing and analysis, potentially ensuring consistent performance across time points and facilitating regulatory compliance for clinical applications.

An alternative embodiment may implement a tiered analytical approach that performs rapid preliminary analysis on each new sample to provide immediate insights, followed by more comprehensive reanalysis of the cumulative dataset to extract maximum information. This approach may balance the need for timely results with the benefits of integrated analysis, potentially providing clinically actionable information on different timescales.

The embodiment of the data integration component may include specialized methods for handling changes in sequencing technologies or reference genomes over extended monitoring periods. These approaches may enable seamless incorporation of data generated with newer platforms or aligned to updated reference assemblies, potentially ensuring that longitudinal monitoring can continue even as methodologies evolve over time.

In some implementations, the system may incorporate automated quality assessment algorithms that evaluate each new sequencing dataset for consistency with established baseline characteristics for that subject. These approaches may flag potential sample mix-ups, contamination events, or technical failures that could compromise longitudinal analysis, potentially ensuring the integrity of time-series genomic data.

The embodiment of the reporting system may include specialized visualization tools that highlight emergence, expansion, and extinction events in the evolving genomic landscape. These interfaces may employ intuitive graphical representations to communicate complex temporal patterns to clinicians or researchers without requiring specialized bioinformatics expertise, potentially enhancing the interpretability and utility of additive sequencing results in practical applications.

### INDUSTRIAL APPLICATION

The additive genomic sequencing technology has significant industrial applications across healthcare and biotechnology sectors. In pharmaceutical development, it enables more sensitive detection of drug-induced genomic changes, accelerating clinical trials and reducing development costs. For clinical diagnostics companies, it offers enhanced testing capabilities for rare variants and somatic mutations, particularly valuable in oncology and rare disease monitoring. The technology can be integrated into existing sequencing platforms to provide value-added services. Biotechnology firms can leverage this approach for longitudinal monitoring of engineered cell lines and gene therapies, while research institutions can employ it for high-resolution population genomics studies with temporal dimensions.

### REFERENCES

1. Brlek P, BulićL, Brac̆ićM, ProjićP, S̆karo V, Shah N, Shah P, Primorac D. Implementing Whole Genome Sequencing (WGS) in Clinical Practice: Advantages, Challenges, and Future Perspectives. Cells. 2024 March 13;13(6):504. doi:10.3390/cells 13060504. PMID: 38534348; PMCID:PMC10969765.
2. Wetterstrand, K. A. (2022). “DNA sequencing costs: Data from the NHGRI Genome Sequencing Program.” NHGRI Genome Program.
3. Sedlazeck, F. J., et al. (2018). “Accurate detection of complex structural variations using single-molecule sequencing.” Nature Methods. DOI: [10.1038/s41592-018-0001-7] (https://doi.org/10.1038/s41592-018-0001-7)
4. Vogelstein, B., et al. (2013). “Cancer genome landscapes.” Science. DOI: [10.1126/science.1235122] (https://doi. org/10.1126/science.1235122)
5. Gerstung, M., et al. (2020). “The evolutionary history of 2,658 cancers.” Nature. DOI: [10.1038/s41586-019-1907-7] (https://doi.org/10.1038/s41586-019-1907-7)
6. Stephens, Z. D., et al. (2015). “Big Data: Astronomical or genomical?” PLOS Biology. DOI:[10.1371/journal.pbio.1002195] (https://doi.org/10.1371/journal.pbio.1002195)
7. DePristo, M. A., et al. (2011). “A framework for variation discovery and genotyping using next-generation DNA sequencing data.” Nature Genetics. DOI:[10.1038/ng.806] (https://doi.org/10.1038/ng.806)
8. Illumina. (2023). DRAGEN Bio-IT Platform. Retrieved from https://www.illumina.com/products/by-type/informatics-products/dragen-bio-it-platform.html
9. Li, H. (2013). “Aligning sequence reads, clone sequences, and assembly contigs with BWA-MEM.” arXiv Preprint arXiv: 1303.3997.
10. Picard Toolkit. Broad Institute. Available at: [https://broadinstitute.github.io/picard/] (https://broadinstitute.github.io/picard/)
11. GATK Best Practices Team. (2022). “GATK Documentation.” Broad Institute. Available at: [https://gatk.broadinstitute.org] (https://gatk.broadinstitute.org)
12. Tarazona, S., et al. (2015). “Differential expression in RNA-seq: A matter of depth.” Genome Research. DOI: [10.1101/gr.191861.115] (https://doi.org/10.1101/gr.191861.115)
13. McKenna, A., et al. (2010). “The Genome Analysis Toolkit: A MapReduce framework for analyzing next-generation DNA sequencing data.” Genome Research. DOI: [10.1101/gr.107524.110] (https://doi.org/10.1101/gr.107524.110)
14. Poplin, R., et al. (2018). “Scaling accurate genetic variant discovery to tens of thousands of samples.” bioRxiv
15. Danecek, P., et al. (2011). “The variant call format and VCFtools.” Bioinformatics. DOI: [10.1093/bioinformatics/btr330] (https://doi.org/10.1093/bioinformatics/btr330)
16. Gilissen, C., et al. (2014). “Genome sequencing identifies major causes of severe intellectual disability.” Nature. DOI: [10.1038/nature 13394] (https://doi.org/10.1038/nature13394)
17. Shendure, J., et al. (2017). “DNA sequencing at 40: Past, present, and future.” Nature. DOI: [10.1038/nature24286] (https://doi.org/10.1038/nature24286)
18. Mardis, E. R. (2017). “DNA sequencing technologies: 2006-2016.” Nature Protocols. DOI: [10.1038/nprot.2016.182] (https://doi.org/10.1038/nprot.2016.182)
19. Kitzman J O, Mackenzie A P, Adey A, Hiatt J B, Patwardhan R P, Sudmant P H, Ng S B, Alkan C, Qiu R, Eichler E E, Shendure J. Haplotype-resolved genome sequencing of a Gujarati Indian individual. Nat Biotechnol. 2011 Jan; 29(1): 59-63. doi: 10.1038/nbt.1740. Epub 2010 December 19. Erratum in: Nat Biotechnol. 2011 May; 29(5): 459. PMID: 21170042; PMCID: PMC3116788.
20. Hartl, D. L., Clark, A. G. (2007). Principles of Population Genetics. 4th ed., Sinauer Associates.
21. Gymrek, M., et al. (2013). “Identifying personal genomes by surname inference.” Science. DOI: [10.1126/science.1229566] (https://doi.org/10.1126/science.1229566)
22. Van der Auwera, G. A., O'Connor, B. (2020). Genomics in the Cloud: Using Docker, GATK, and WDL in Terra. O'Reilly Media.
23. Choi, M., et al. (2009). “Genetic diagnosis by whole exome capture and massively parallel DNA sequencing.” Proceedings of the National Academy of Sciences. DOI: [10.1073/pnas.0910672107] (https://doi.org/10.1073/pnas.0910672107)
24. Cummings, B. B., et al. (2020). “Transcript expression-aware annotation improves rare variant interpretation.” Nature. DOI: [10.1038/s41586-020-2329-2] (https://doi.org/10.1038/s41586-020-2329-2)
25. Wandelt, S., Rheinländer, A., Bux, M. et al. Data Management Challenges in Next Generation Sequencing. Datenbank Spektrum 12, 161-171 (2012). https://doi.org/10.1007/s13222-012-0098-2
26. Wan, J. C. M., et al. (2017). “Liquid biopsies come of age: Towards implementation of circulating tumor DNA.” Nature Reviews Cancer. DOI: [10.1038/nrc.2017.7] (https://doi.org/10.1038/nrc.2017.7)
27. Philip Ewels, Måns Magnusson, Sverker Lundin, Max Käller, MultiQC: summarize analysis results for multiple tools and samples in a single report, Bioinformatics, Volume 32, Issue 19, October 2016, Pages 3047-3048, https://doi.org/10.1093/bioinformatics/btw354
28. Alix-Panabières C, Pantel K. Liquid Biopsy: From Discovery to Clinical Application. Cancer Discov. 2021 Apr; 11(4): 858-873. Doi: 10.1158/2159-8290.CD-20-1311. PMID: 33811121.
29. Burton H, Jackson C, Abubakar I. The impact of genomics on public health practice. Br Med Bull. 2014 Dec; 112(1):37-46. Doi: 10.1093/bmb/ldu032. Epub 2014 November 3. PMID: 25368375; PMCID: PMC7110005.
30. Johnson, J. A., et al. (2013). “Pharmacogenetics: potential for individualized drug therapy through genetics.” Trends in Genetics. DOI: [10.1016/j.tig.2013.01.005] (https://doi.org/10.1016/j.tig.2013.01.005)
31. Wensing, A. M., et al. (2019). “2019 update of the drug resistance mutations in HIV-1.” Topics in Antiviral Medicine. PMID:31794204
32. Zhu, Q., Zhao, X., Zhang, Y. et al. Single-cell multi-omics reveal intra-cell-line heterogeneity across human cancer cell lines. Nat Commun 14, 8170 (2023). https://doi.org/10.1038/s41467-023-43991-9
33. Hasin, Y. et al. (2017). “Multi-omics approaches to disease.” Genome Biology. DOI: [10.1186/s13059-017-1215-1] (https://doi.org/10.1186/s13059-017-1215-1)
34. Koboldt, D. C. Best practices for variant calling in clinical sequencing. Genome Med 12, 91 (2020). https://doi.org/10.1186/s13073-020-00791-w
35. English, A. C., et al. (2015). “Assessing structural variation in a personal genome—towards a human reference diploid genome.” BMC Genomics. DOI: [10.1186/s12864-015-1479-3](https://doi.org/10.1186/s12864-015-1479-3)
36. Chaisson, M. J. P., et al. (2019). “Multi-platform discovery of haplotype-resolved structural variation in human genomes.” Nature Communications. DOI: [10.1038/s41467-018-08148-z] (https://doi.org/10.1038/s41467-018-08148-z)
37. Check Hayden, E. (2014). “Technology: The $1,000 genome.” Nature. DOI: [10.1038/507294a] (https://doi.org/10.1038/507294a)

What is claimed is:

1. A system for additive genomic analysis comprising:
a sample management component configured to track multiple biological samples collected from a subject at predetermined time intervals;
a sequencing component configured to perform moderate-depth sequencing on each biological sample to generate sequencing data;
a data storage component configured to store sequencing data from each time point;
a computational processing component comprising at least one processor configured to:
align the sequencing data from each time point to a reference genome,
merge aligned sequencing data from multiple time points to generate a cumulative dataset with increased coverage depth,
identify genomic variants using the cumulative dataset, and
track temporal changes in variant allele frequencies across time points; and
an output component configured to generate reports that include both cumulative coverage analysis and longitudinal tracking of genomic variations.

2. The system of claim 1, wherein the predetermined time intervals comprise weekly intervals resulting in 52 sequencing events per year.

3. The system of claim 1, wherein the sequencing component is configured to perform whole-genome sequencing at approximately 10X coverage depth per time point.

4. The system of claim 1, wherein the sequencing component is configured to perform whole-exome sequencing at approximately 30X coverage depth per time point.

5. The system of claim 1, wherein the computational processing component further comprises a hardware-accelerated pipeline utilizing DRAGEN technology.

6. The system of claim 1, wherein the computational processing component is further configured to implement batch effect correction techniques to minimize variability between sequencing runs.

7. A method for additive genomic sequencing comprising:
obtaining a plurality of biological samples from a subject at predetermined time intervals;
performing sequencing on each of the plurality of biological samples to generate a plurality of sequencing datasets, each sequencing dataset having a defined coverage depth;
aligning each of the sequencing datasets to a reference genome to generate a plurality of aligned datasets;
merging the plurality of aligned datasets to generate a cumulative sequencing dataset having a cumulative coverage depth, wherein the cumulative coverage depth is substantially greater than the defined coverage depth of any individual sequencing dataset; and
analyzing the cumulative sequencing dataset to identify genomic variants present in the subject.

8. The method of claim 7, wherein the predetermined time intervals are selected from the group consisting of daily, weekly, monthly, and quarterly intervals.

9. The method of claim 7, wherein performing sequencing comprises performing low-pass whole-genome sequencing at approximately 10X coverage per time point.

10. The method of claim 7, wherein performing sequencing comprises performing high-pass whole-exome sequencing at approximately 30X coverage per time point.

11. The method of claim 7, further comprising performing quality control checks at each time point to ensure consistency across sequencing runs.

12. The method of claim 7, wherein analyzing the cumulative sequencing dataset comprises detecting rare variants with allele frequencies below detection thresholds of individual sequencing datasets.

13. The method of claim 7, further comprising tracking the emergence and evolution of somatic mutations across the predetermined time intervals.

14. A computer-implemented method for integrating temporal genomic data, comprising:
receiving a plurality of sequencing datasets generated from biological samples collected from a subject at different time points;
processing each of the sequencing datasets through a standardized bioinformatics pipeline to generate aligned read data and variant call data for each time point;
implementing batch effect correction to minimize technical variations between the sequencing datasets collected at different time points;
aggregating the aligned read data from each time point to generate a cumulative coverage dataset;
performing variant calling on the cumulative coverage dataset to identify genomic variants;
tracking allele frequencies of the identified genomic variants across the different time points; and
generating a longitudinal profile of genomic changes in the subject based on temporal variations in the identified genomic variants.

15. The computer-implemented method of claim 14, wherein the standardized bioinformatics pipeline comprises demultiplexing, quality control, alignment, duplicate removal, and base quality recalibration steps.

16. The computer-implemented method of claim 14, wherein implementing batch effect correction comprises applying ComBat methodology to reduce variability stemming from library preparation or platform changes.

17. The computer-implemented method of claim 14, further comprising storing intermediate data files in a distributed cloud-based storage system with version control.

18. The computer-implemented method of claim 14, wherein performing variant calling comprises utilizing a combination of GATK HaplotypeCaller and FreeBayes algorithms.

19. The computer-implemented method of claim 14, further comprising applying statistical methods to identify significant temporal trends in variant allele frequencies.

20. The computer-implemented method of claim 14, further comprising integrating the longitudinal profile of genomic changes with clinical data to identify correlations between genetic variations and disease progression.
